(19) United States
(12) Patent Application Publication
(10) Pub. No.: US 2009/0068727 A1
(43) Pub. Date: Mar. 12, 2009

(54) CLOSED SYSTEM, SHALLOW CHANNEL PHOTOBIOREACTOR

(76) Inventor: Greg Karr, St. George, KS (US)

Correspondence Address: SCHWEGMAN, LUNDBERG & WOESSNER, P.A., P.O. BOX 2938, MINNEAPOLIS, MN, 55402, US

(21) Appl. No.: 12/199,417

(22) Filed: Aug. 27, 2008

Related U.S. Application Data
(60) Provisional application No. 60/968,490, filed on Aug. 28, 2007.

(51) Int. Cl. C12M 1/00

(52) U.S. Cl. 435/292.1

(57) ABSTRACT

The present subject matter provides examples of a photobioreactor for producing biomass material using a continuous flow of liquid growth media, the apparatus comprising a channel including a bottom portion, a first sidewall portion connected to the bottom portion, and a second sidewall portion connected to the bottom portion opposite the first sidewall portion, wherein a length of the channel is at least twice as long as a width of the channel; the length of the channel is measured along the bottom portion of the channel and parallel to the first and second sidewall portions of the channel, and the width is measured as a shortest distance between and perpendicular to the first and second sidewall portions of the channel, a transparent cover adapted to provide a seal with the first sidewall and second sidewall opposite the bottom portion along a substantial length of the channel, a liquid growth media input at a first end of the channel, a biomass and liquid media output for harvesting biomass material at a second end of the channel, and a gas backflow barrier positioned adjacent the transparent cover and between the first and second sidewall portions, the gas backflow barrier adapted to limit gas from passing from an interior portion of the channel through the liquid growth media input when the liquid growth media is flowing.

### CLAIM OF PRIORITY AND RELATED APPLICATION

This application claims the benefit under 35 U.S.C. 119(e) of U.S. Provisional Patent Application Ser. No. 60/968,490, filed Aug. 28, 2007, the entire disclosure of which is hereby incorporated by reference in its entirety.

### TECHNICAL FIELD

This application relates to photobioreactors and more particularly to continuous flow photobioreactors.

### BACKGROUND

Photobioreactors provide an artificial environment for growing biomass materials, including algae, for a variety of research and commercial uses. Such uses include, but are not limited, to development and production of alternative fuel sources. The design of a photo bioreactor depends on the application for which the biomass is produced and, therefore, must consider the specific requirements of the biological system used. Exposure of the biomass material to adequate light, nutrients and carbon dioxide are a few of the basic requirements applicable to most bioreactor designs.

### SUMMARY

The subject matter of this document provides an efficient and low cost photobiorector apparatus and method for high capacity production of biomass material. In various embodiments, the apparatus provides a closed system, shallow channel photo-bioreactor with an input and an output to allow continuous production of biomass material. In various embodiments, the photobioreactor allows light to access the growing biomass material using a transparent cover exposed to either natural or artificial light. In various embodiments, at least one gas discharge tube extending substantially the full length of the photobioreactor is used to expose the biomass material to carbon dioxide (CO₂) rich gas emitted through perforations extending the length of the gas discharge tube. One embodiment of the subject matter of this document provides a photobioreactor system using multiple, closed system, shallow channel bioreactors for use as a high capacity method of biomass production.

This Summary is an overview of some of the teachings of the present application and is not intended to be an exclusive or exhaustive treatment of the present subject matter. Further details about the present subject matter are found in the detailed description. The scope of the present invention is defined by the appended claims and their equivalents.

### BRIEF DESCRIPTION OF DRAWINGS

FIG. 1A is a side view of a photobioreactor according to one embodiment of the present subject matter.

FIG. 1B shows a prospective view of a photobioreactor according to one embodiment of the present subject matter.

FIG. 2 is a cross-section of a closed system, shallow channel photobioreactor according to one embodiment of the present subject matter.

FIG. 3 illustrates a detailed view of a gas discharge tube near the transparent cover of a photobioreactor according to one embodiment of the present subject matter.

FIG. 4 shows a side view of a photobioreactor with gas release ports according to one embodiment of the present subject matter.

FIG. 5 illustrates a photobioreactor system according to one embodiment of the present subject matter.

FIG. 6 is a flowchart for producing biomass material with a closed, shallow channel photobioreactor according to one embodiment of the present subject matter.

### DETAILED DESCRIPTION

The following detailed description refers to subject matter in the accompanying drawings which show, by way of illustration, specific aspects and embodiments in which the present subject matter may be practiced. These embodiments are described in sufficient detail to enable those skilled in the art to practice the present subject matter. References to “an”, “one”, or “various” embodiments in this disclosure are not necessarily to the same embodiment, and such references contemplate more than one embodiment. The following detailed description is, therefore, not to be taken in a limiting sense, and the scope is defined only by the appended claims, along with the full scope of legal equivalents to which such claims are entitled.

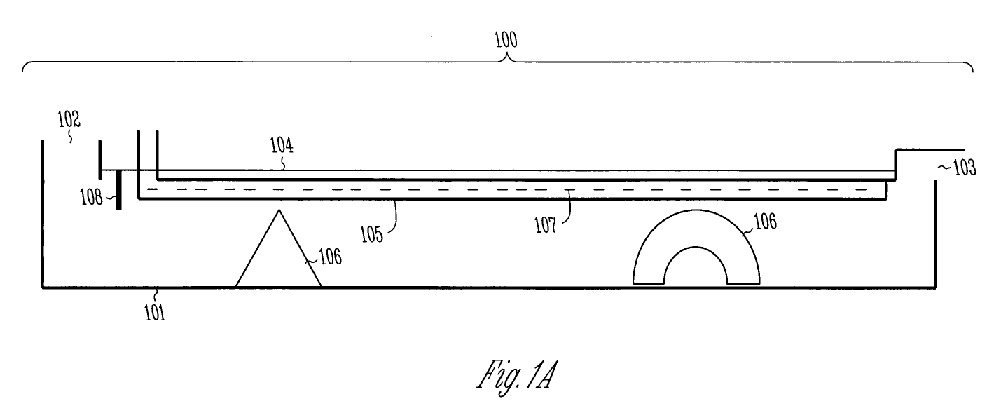

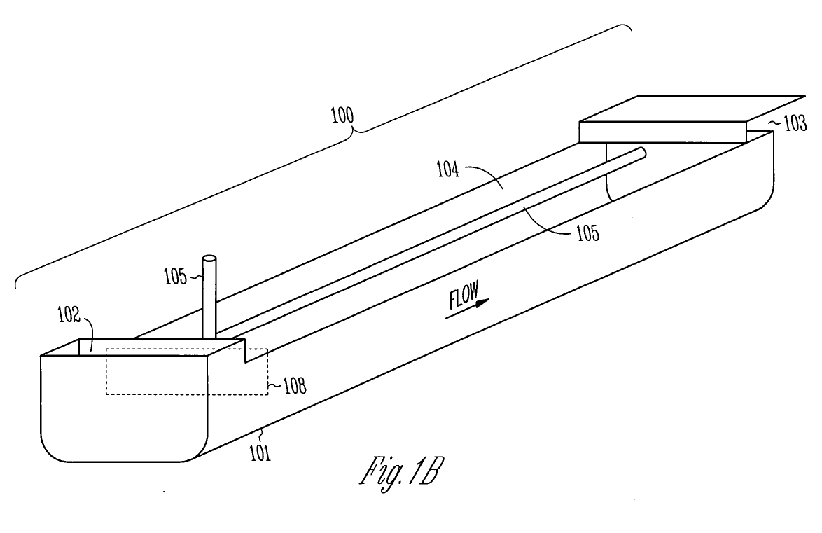

FIGS. 1A and 1B show a closed system, shallow channel photobioreactor 100 according to one embodiment of the present subject matter. FIGS. 1A and 1B show a channel 101 with a liquid growth input 102 at one end, a liquid and biomass output 103 at the other end, a transparent cover 104, a gas discharge tube 105 and various liquid flow barriers 106. In various embodiments, the length of the channel from the liquid growth media to the liquid and biomass output is a substantially greater distance than the width of the channel as measured between the channel sidewalls. For example, the channel length should be at least 2 times greater than the channel width. In various embodiments, the channel length is more than 1800 times greater than the channel width. In various embodiments, the channel has a U-shaped cross-section, but is not so limited. In various embodiments the channel is formed of rigid water tight materials such as plastic, metal or concrete. In various embodiments, an earthen channel is lined with a poly film, such as polyethylene, or membrane material, such as Duro-Last® roofing membrane made by Duro-Last® Roofing, Inc.

The cover 104 of the channel is a flat, rigid, transparent sheet material and is connected to the sidewalls of the channel so as to provide a water-tight seal between the cover and the sidewalls. Rigid materials from which the cover may be made include, for example, plastic polymers and glass. In various embodiments, the cover material is a thin transparent film and is supported during operation of the photobioreactor by the liquid growth media inside the photobioreactor channel. In some embodiments, the cover is weatherproof and connected to the channel such that it resists damage due to wind, rain, hail or combinations thereof.

A gas discharge tube 105 discharges gases to promote growth of the biomass material within the photobioreactor channel. The gas is discharged through openings 107 in the gas discharge tube 105. The openings 107 in the gas discharge tube 105 extend the length of the tube and are positioned at various spacing with respect to each other. In various embodiments, the gas discharge tube extends along substantially the entire length of the channel and is located near the cover 104 of the channel. In various embodiments, a channel contains a plurality of gas discharge tubes extending substantially the entire length of the photobioreactor channel. A gas backflow barrier plate 108 is located near the liquid growth input 102 to prevent gas bubbles, discharged from the gas discharge tube 105, from exiting the channel at the liquid growth input 102. The gas barrier plate 108 ensures the gas bubbles move with the flow of the liquid growth media and biomass through the entire channel allowing as much gas as possible to be absorbed into the liquid growth media. Any unabsorbed gas or gas produced through the biomass growth process, is allowed to exit the covered channel at the liquid and biomass output 103, minimizing backpressure. In various embodiments, the gas discharge tubes are connected to a gas source in which the gas is rich in carbon dioxide (CO₂).

Operation of a photobioreactor reactor according to one embodiment of the present subject matter, commences with filling the channel completely to the top cover with liquid growth media. The channel is orientated such that it is longitudinally horizontal, less than 1% incline or decline, and the transparent cover will receive adequate light. The liquid growth media is then seeded with biomass material. Growth is encouraged by exposing the biomass material to light and CO₂ rich gas. The light is provided either naturally, such as the sun, or artificially, through the transparent cover of the channel. The CO₂ rich gas is provided through at least one perforated gas discharge tube. After the initially seeded biomass material reaches a predetermined density, the process becomes continuous by providing a continuous flow of liquid growth media through the liquid growth input 102 and allowing liquid and biomass material to exit the photobioreactor through the liquid and biomass outlet 103. In various embodiments, flow of the liquid growth media is monitored and controlled using level controls located at the liquid growth input 102 and the liquid and biomass output 103.

To maximize the growth of the biomass material, various embodiments provide features to reduce stagnation of the movement of the biomass material through the channel. Stagnated movement of the biomass material results in a minimal amount of gas being absorbed by the liquid growth media. Stagnated movement of the biomass material also blocks light to biomass material not adjacent to the transparent cover, thus reducing the overall growth potential of the channel. Additionally, stagnated movement reduces the availability of nutrients to biomass material not adjacent to the flow of liquid growth media. In various embodiments, flow barriers 106 are provided in the channel to induce turbulence of the liquid growth media by diverting the flow of the media in various directions. The turbulence disrupts stagnant biomass material and creates a continuous and even mixture of the liquid growth media and biomass material throughout the length of the photobioreactor channel. In various embodiments, actuated devices, such as impellers, are used to create turbulence and mix the liquid growth media and the biomass material. An efficient mixing of the liquid growth media flow and the biomass material provides more probability that all the biomass material will be exposed to adequate light, gas and nutrients for sufficient growth as the biomass moves with the flow of liquid growth media from the liquid growth input 102 to the liquid and biomass output 103, where the biomass is captured for further processing.

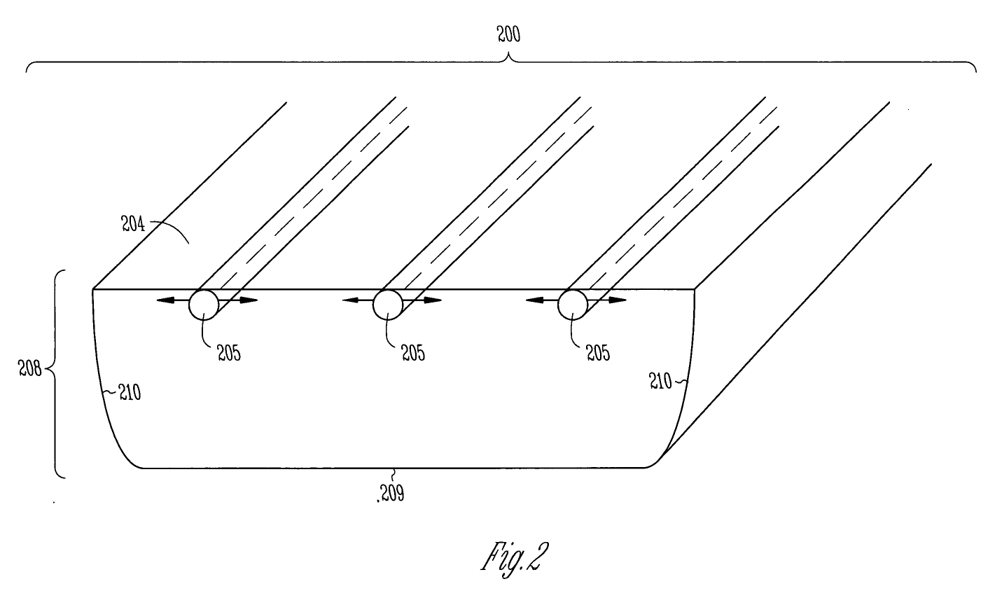

FIG. 2 is a cross-section of a closed system, shallow channel photobioreactor 200 according to one embodiment of the present subject matter. FIG. 2 illustrates a U-shaped channel 208 with a bottom portion 209, two adjoining side wall portions 210, a transparent cover 204 and a plurality of perforated gas discharge tubes 205 positioned near the transparent cover 204. In various embodiments, each gas discharge tube 205 is capped at one end and connected to a CO₂ rich gas source at the other. Examples of CO₂ rich sources of gas include gases produced from fuel combustion, fermentation processes and digestation processes.

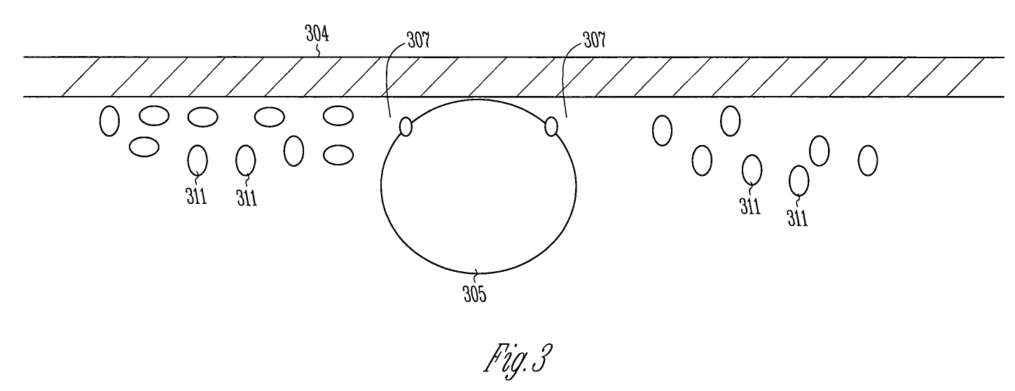

FIG. 3 illustrates a detailed view of a gas discharge tube 305 near the transparent cover 304 of a photobioreactor according to one embodiment of the present subject matter. FIG. 3 shows a gas discharge tube 305 with openings 307 directing the discharge of CO₂ rich gas bubbles 311 toward the transparent cover 304 of the photobioreactor channel. In addition to introducing CO₂ for absorption by the liquid growth media, the gas discharge tubes 305 direct the gas bubbles 311 at the transparent cover 304 in a direction that creates shear forces that prevent and reduce the amount of biomass material accumulating on the cover 304. Biomass material that accumulates on the transparent cover 304 reduces the amount of light available to other biomass material, therefore reducing the efficiency of the photobioreactor. Additionally, the turbulence of the liquid growth media created by the discharged gas bubbles 311 near the transparent cover 304 provides vertical mixing of the biomass material and the liquid growth media providing opportunity for the liquid growth media to circulate into close proximity of the light coming through the transparent cover 304, thus providing light energy to a greater volume of liquid media.

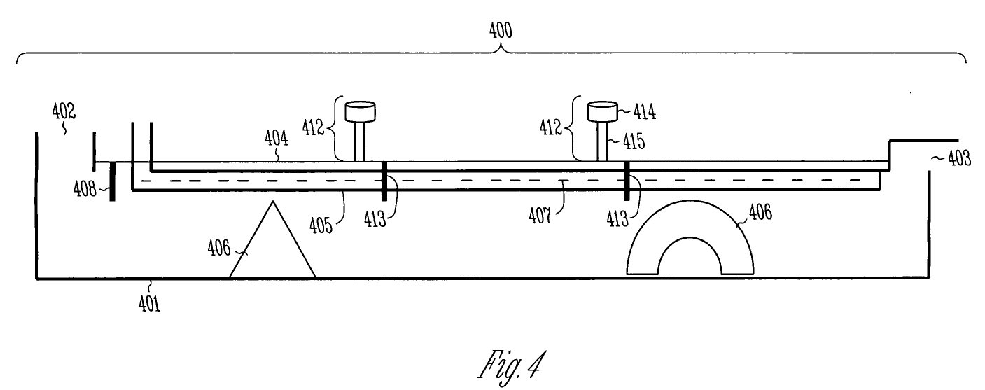

FIG. 4 shows a side view of a photobioreactor 400 with gas release ports according to one embodiment of the present subject matter. FIG. 4 shows a channel 401 with a liquid growth input 402 at one end, a liquid and biomass output 403 at the other end, a transparent cover 404, a gas discharge tube 405 and various liquid flow barriers 406. In various embodiments, the length of the channel from the liquid growth media to the liquid and biomass output is a substantially greater distance than the width of the channel as measured between the channel sidewalls. For example, the channel length should be at least 2 times greater than the channel width. In various embodiments, the channel length is more than 1800 times greater than the channel width. In various embodiments, the channel has a U-shaped cross-section, but is not so limited.

The illustrated embodiment of FIG. 4 includes one or more gas release ports 412 and gas release backflow barriers 413 along the length of the transparent cover 404. The illustrated gas release ports 412 include a gas release valve 414 mounted on top of a gas vent 415, such as a pipe, for example. In various embodiment, the gas release valve 414 is a one-way valve allowing gas from inside the vent to escape without allowing any ambient air, including potential airborne contaminants to enter the bioreactor. The gas release ports 412 prevent a continuous layer of gas to form above the biomass material and growth media. The gas release ports 412 assure the underside of the transparent cover 404 is in contact with the biomass and growth media. The turbulance of the moving biomass material, growth media and gas bubbles at or near the transparent cover provides shear forces that reduce the adhesion of biomass material to the transparent cover. As a result, more favorable production conditions exist as light energy entering the chamber is not reduced by material accumulating to the transparent cover. Additionally, the gas release ports allow oxygen generated from the growth of the biomass material and of little benefit to the continued growth of the biomass material to escape the bioreactor. In various embodiments, such as the embodiment illustrated, gas backflow barriers 413 are positioned down-flow from each gas release port 412 to increase the residence time of freshly discharged CO₂. The gas backflow barriers 413 reduce the release of CO₂ discharged near each gas release port from the one or more gas discharge tubes 405. In various embodiments, the gas release ports 412 are spaced evenly from each other along the length of the bioreactor channel. The number of gas release ports is determined by the length of the bioreactor and the amount of gas produced by the biomass material. In various embodiments, the gas release vents 415 extend to a height above the transparent cover 404 equal to, or greater than, the height of the fluid level of the liquid growth media.

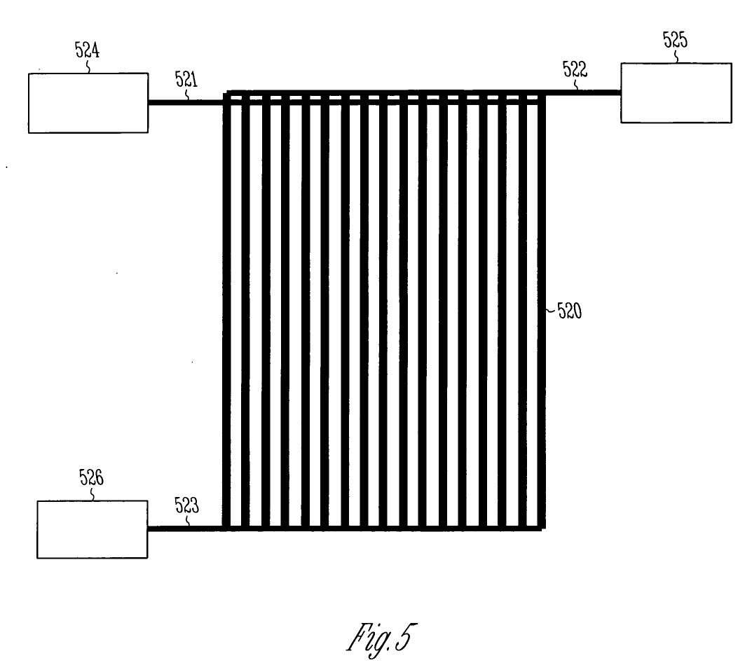

FIG. 5 illustrates a photobioreactor system according to one embodiment of the present subject matter. The system includes a plurality of closed, shallow channel photobioreactors 520 as described above. In various embodiments, the photobioreactors 520 are positioned parallel to each other and spaced to allow maintenance while at the same time minimizing area occupied by the reactors to reduce capital costs. In the illustrated embodiment, the gas discharge tubes of each channel are connected to a gas source 524 using a gas distribution manifold 521. In the illustrated embodiment, the liquid growth input of each channel is connected to a source of liquid growth media 525 using a liquid growth media distribution manifold 522. In various embodiments, flow of the liquid growth media is monitored and controlled using level controls located at the liquid growth input and the liquid and biomass output. In various embodiments, the flow rate of the liquid growth media and the discharge gas are controlled using manual controls. In various embodiments, the flow rate of the liquid growth media and the discharge gas are controlled automatically using a controller and automatic controls. In various embodiments, the flow rate of the liquid growth media and the discharge gas are controlled using both manual and automatic controls. In various embodiments, the flow of the liquid growth media at each channel is controlled at a rate sufficient to sustain continuous biomass production at the liquid and biomass output while at the same time not exceeding a flow rate that would provide insufficient residence time of the biomass material resulting in low production rates in proportion to the liquid growth media input or a rate that would eventually deplete biomass material from the channel. The illustrated embodiment of a photobioreactor system according to the present subject matter of FIG. 5 shows the liquid and biomass output of each channel of the system connected to common biomass collection system 523. The collection system allows for collection and transport of harvested biomass to a common location 526 for further processing and/or distribution.

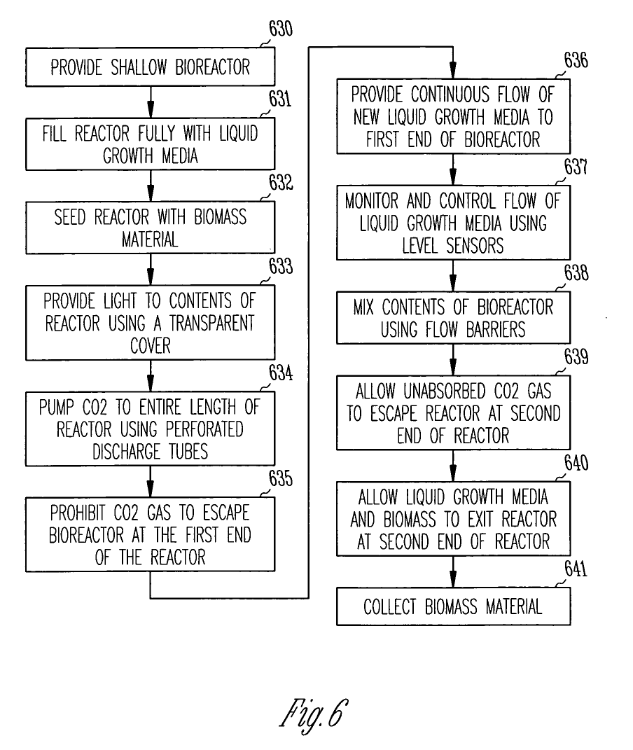

FIG. 6 is a flowchart for producing biomass material with a closed, shallow channel photobioreactor according to one embodiment of the present subject matter. The process is initiated by providing a lengthy, closed, shallow channel photobioreactor 630 and then fully filling the channel with liquid growth media 631 and seeding the media with biomass material 632. In various embodiments, a shallow channel is one between 4 inches and 5 feet in depth. Growth of the seeded biomass material is encouraged by providing light 633 and CO₂ rich gas to the photobioreactor 634. In various embodiments, light is transmitted to the chamber through a clear channel cover exposed to natural light. In various embodiments, the clear channel cover is exposed to artificial light. In various embodiments, CO₂ rich gas is added to the channel using at least one gas discharge tube extending the length of the channel and located inside the channel and adjacent the transparent cover. CO₂ gas is prohibited from exiting a first end of the photobioreactor using a gas barrier near the first end of the photobioreactor 635.

After the seeded biomass material has grown to a predetermined density, the process becomes continuous by providing a flow of liquid growth media to the first end of the photobioreactor 636. Adding new liquid growth media to the first end of the channel creates hydraulic pressure and subsequent flow through the channel to a second end of the channel. In various embodiments, the flow rate of the photobioreactor is controlled using level sensors located at the first and second ends of the channel 637. As the biomass material proceeds with the flow of the liquid growth media from the first end of the photobioreactor to the second end of the photobioreactor, the liquid growth media and biomass material are mixed together using flow barriers positioned along the length of the photobioreactor 638. Unabsorbed gas is allowed to exit the reactor at the second end of the photobioreactor 639. As a sufficient positive pressure and flow of growth material is established in the photobioreactor, excess liquid and biomass material are allowed to exit the reactor at the second end of the photobioreactor 640. As the biomass material exits the photobioreactor, it is collected for further processing and/or distribution 641.

This application is intended to cover adaptations or variations of the present subject matter. It is to be understood that the above description is intended to be illustrative, and not restrictive. The scope of the present subject matter should be determined with reference to the appended claims, along with the full scope of equivalents to which such claims are entitled.

What is claimed is:

1. A photobioreactor for producing biomass material using a flow of liquid growth media, the apparatus comprising:
a channel including,
a bottom portion;
a first sidewall portion connected to the bottom portion; and
a second sidewall portion connected to the bottom portion opposite the first sidewall portion,
wherein a length of the channel is at least twice as long as a width of the channel, wherein the length of the channel is measured along the bottom portion of the channel and parallel to the first and second sidewall portions of the channel, and the width is measured as a shortest distance between and perpendicular to the first and second sidewall portions of the channel;
a transparent cover adapted to provide a seal with the first sidewall and second sidewall opposite the bottom portion along a substantial length of the channel;
a liquid growth media input at a first end of the channel;
a biomass and liquid media output for harvesting biomass material at a second end of the channel; and
a gas backflow barrier positioned adjacent the transparent cover and between the first and second sidewall portions, the gas backflow barrier adapted to limit gas from passing from an interior portion of the channel through the liquid growth media input when the liquid growth media is flowing.

2. The bioreactor of claim 1, further comprising one or more gas discharge tubes extending through the liquid growth media along a substantial length of the channel, the gas discharge tubes adapted to discharge carbon dioxide (CO₂) rich gas into the liquid growth media.

3. The bioreactor of claim 2, wherein at least one of the one or more gas discharge tubes is located adjacent the transparent cover and is adapted to discharge CO₂ rich gas toward the cover to reduce accumulation of biomass material on the cover.

4. The bioreactor of claim 2, wherein the transparent cover includes one or more gas release ports adapted to eliminate a continuous gas film from forming between the cover and the liquid growth media.

5. The bioreactor of claim 4, wherein the one or more gas release ports include a vent having a first end coupled to the transparent cover and extending away from the channel

6. The bioreactor of claim 5, wherein the one or more gas release ports further include a gas release valve coupled to a second end of the vent and the gas release valve is positioned above a highest level of the liquid growth media in the photobioreactor.

7. The bioreactor of claim 4, further comprising one or more gas release backflow barriers adapted to prevent premature release of CO₂ from the liquid growth media, wherein each gas release backflow barrier is positioned adjacent to and down-flow from one of the one or more gas release ports.

8. The bioreactor of claim 1, further comprising a plurality of flow barriers attached to the channel, the flow barriers adapted to perturb the continuous flow of the liquid growth media to cycle the biomass material toward the transparent cover.

9. The bioreactor of claim 1, wherein the cover is at least partially supported using the liquid growth media.

10. The bioreactor of claim 8, wherein the transparent cover is rigid.

11. The bioreactor of claim 8, wherein the transparent cover is a film.

12. A photobioreactor for producing biomass material using a continuous flow of liquid growth media, the apparatus comprising:
a channel including,
a bottom portion;
a first sidewall portion connected to the bottom portion; and
a second sidewall portion connected to the bottom portion opposite the first sidewall portion,
wherein a length of the channel is at least twice as long as a width of the channel; the length of the channel is measured along the bottom portion of the channel and parallel to the first and second sidewall portions of the channel, and the width is measured as a distance between and perpendicular to the first and second sidewall portions of the channel;
a transparent cover adapted to provide a seal with the first sidewall portion and second sidewall portion opposite the bottom portion along a substantial length of the channel;
a liquid growth media input at a first end of the channel;
a biomass and liquid media output for harvesting biomass material at a second end of the channel;
one or more gas release ports adapted to eliminate a continuous gas film from forming between the cover and the liquid growth media; and
one or more gas discharge tubes adapted to discharge CO₂ rich gas into the liquid growth media.

13. The bioreactor of claim 11, wherein the one or more gas discharge tubes extend through the liquid growth media along a substantial length of the channel.

14. The bioreactor of claim 11, wherein at least one of the one or more gas discharge tubes is located adjacent the transparent cover and is adapted to discharge CO₂ rich gas toward the cover to reduce accumulation of biomass material on the cover.

15. The bioreactor of claim 11, wherein the one or more gas release ports include
a vent having a first end coupled to the transparent cover and extending away from the channel.

16. The bioreactor of claim 14, wherein the one or more gas release ports further includes a gas release valve coupled to a second end of the vent and the gas release valve is positioned above a highest level of the liquid growth media in the photobioreactor.

17. The bioreactor of claim 11, further comprising one or more gas release backflow barriers adapted to prevent the premature release of CO₂ from the liquid growth media, wherein each gas release backflow barrier is positioned adjacent to and down-flow from one of the one or more gas release ports.

18. The bioreactor of claim 11, further comprising a plurality of flow barriers attached to the channel, the flow barriers adapted to perturb the continuous flow of the liquid growth media to cycle the biomass material toward the transparent cover.

19. The bioreactor of claim 11, wherein the cover is at least partially supported using the liquid growth media.

20. The bioreactor of claim 18, wherein the transparent cover is rigid.

21. The bioreactor of claim 18, wherein the transparent cover is a film.
